(19) United States
(12) Patent Application Publication
(10) Pub. No.: US 2023/0172655 A1
(43) Pub. Date: Jun. 8, 2023

(54) MULTIFUNCTIONAL ACTIVATION FOR IN-LINE VESSEL SEALER

(71) Applicant: Covidien LP, Mansfield, MA (US)

(72) Inventors: Rajanikanth Mandula, Hyderabad (IN); Ravi Sekhar Gutti, Hyderabad (IN)

(21) Appl. No.: 18/075,470

(22) Filed: Dec. 6, 2022

Related U.S. Application Data
(60) Provisional application No. 63/286,581, filed on Dec. 7, 2021.

(51) Int. Cl. A61B 18/14; A61B 17/29

(52) U.S. Cl. CPC A61B 18/1445; A61B 17/29; A61B 2018/1455; A61B 2018/0063

(57) ABSTRACT

A surgical instrument includes a housing having an elongated shaft with jaw members at a distal end thereof configured to treat tissue upon electrical activation thereof. A first handle is movable relative to the housing to move the jaw members relative to one another to grasp tissue therebetween. A switch is disposed on the housing and configured for activation by the first handle when fully actuated. A slider is disposed in the first handle and is movable between a first position configured to align a switch activation element on the first handle with the switch to activate the jaw members upon full actuation of the first handle and a second position wherein the switch activation element is misaligned with the switch such that full actuation of the first handle does not activate the jaw members.

### CROSS REFERENCE TO RELATED APPLICATION

This application claims the benefit of U.S. Provisional Application Ser. No. 63/286,581 filed Dec. 7, 2021, the entire contents of which being incorporated by reference herein.

### BACKGROUND

### Technical Field

The present disclosure relates generally to the field of surgical instruments. In particular, the disclosure relates to an in-line, endoscopic electrosurgical forceps that is economical to manufacture and is capable of sealing and cutting tissue structures.

### Background of Related Art

Instruments such as electrosurgical forceps are commonly used in open and endoscopic surgical procedures to coagulate, cauterize and seal tissue. Such forceps typically include a pair of jaw members that can be controlled by a surgeon to grasp targeted tissue, such as, e.g., a blood vessel. The jaw members may be approximated to apply a mechanical clamping force to the tissue, and are associated with at least one electrode to permit the delivery of electrosurgical energy to the tissue. The combination of the mechanical clamping force and the electrosurgical energy has been demonstrated to join adjacent layers of tissue captured between the jaw members. When the adjacent layers of tissue include the walls of a blood vessel, sealing the tissue may result in hemostasis, which may facilitate the transection of the sealed tissue. A detailed discussion of the use of an electrosurgical forceps may be found in U.S. Pat. No. 7,255,697 to Dycus et al.

A bipolar electrosurgical forceps typically includes opposed electrodes disposed on clamping faces of the jaw members. The electrodes are charged to opposite electrical potentials such that an electrosurgical current may be selectively transferred through tissue grasped between the electrodes. To effect a proper seal, particularly in relatively large vessels, two predominant mechanical parameters must be accurately controlled; the pressure applied to the vessel, and the gap distance established between the electrodes.

Both the pressure and gap distance influence the effectiveness of the resultant tissue seal. If an adequate gap distance is not maintained, there is a possibility that the opposed electrodes will contact one another, which may cause a short circuit and prevent energy from being transferred through the tissue. Also, if too low a force is applied the tissue may have a tendency to move before an adequate seal can be generated. The thickness of a typical effective tissue seal is optimally between about 0.001 and about 0.006 inches. Below this range, the seal may shred or tear and above this range the vessel walls may not be effectively joined. Closure pressures for sealing large tissue structures preferably fall within the range of about 3 kg/cm² to about 16 kg/cm².

In-line electrosurgical forceps are one common type of electrosurgical instrument which offers the ease of electrically activating the forceps when fully and continuously compressing the same handle used to close the jaw members about tissue. In some instances, the surgeon may simply desire to grasp tissue and not electrically activate the jaw members or manually activate the jaw members after grasping and manipulation of tissue. As such, it would be desirous to manufacturer an in-line electrosurgical forceps that facilitates both grasping tissue and in-line activation independently of one another and/or cotemporaneous with one another depending upon a surgeon's choice.

### SUMMARY

Provided in accordance with the present disclosure is a surgical instrument that includes a housing having a shaft with an end effector assembly disposed at a distal end thereof. The end effector includes first and second jaw members configured to treat tissue upon electrical activation thereof. A first handle is movable relative to the housing and is configured to move one or both of the first or second jaw members relative to the other of the first or second jaw members to grasp tissue therebetween. The first handle includes a switch activation element extending therefrom. A switch is disposed on the housing and is disposed in the actuation path of the first handle, the switch configured for activation by the first handle when the first handle is fully actuated relative to the housing. A slider is disposed in the first handle and is movable between a first, in-line position configured to align the switch activation element with the switch to activate the jaw members upon full actuation of the first handle relative to the housing and a second position wherein the switch activation element is misaligned with the switch such that full actuation of the first handle does not activate the jaw members.

In aspects according to the present disclosure, the switch is operably coupled to a depressible button extending from the housing and is configured to be selectively engaged by the switch activation element of the first handle when the slider is disposed in the first, in-line position.

In aspects according to the present disclosure, the slider is slidable within a channel defined within the first handle.

In aspects according to the present disclosure, the housing includes a second handle depending therefrom configured to support the switch therein. In other aspects according to the present disclosure, the second handle includes an aperture defined therein configured to receive the switch activation element of the first handle upon full actuation of the first handle when the slider is disposed in the second position.

In aspects according to the present disclosure, the housing includes a latch assembly disposed therein configured to releasably lock the first handle relative to the housing when the slider is disposed in the second position. In other aspects according to the present disclosure, the latch assembly is moveable between a first position wherein the latch assembly is aligned with a railway defined within the housing for selectively locking the first handle relative to the housing upon actuation thereof and a second position wherein the latch assembly is misaligned with the railway and the first handle does not releasably lock relative to the housing. In still other aspects according to the present disclosure, the slider includes a rod operably coupled to the latch assembly, the rod configured to move the latch assembly between the first position and the second position upon movement of the slider.

Provided in accordance with the present disclosure is a surgical instrument that includes a housing having a shaft with an end effector assembly disposed at a distal end thereof. The end effector includes first and second jaw members configured to treat tissue upon electrical activation thereof. A first handle is movable relative to the housing and is configured to move one or both of the first or second jaw members relative to the other of the first or second jaw members to grasp tissue therebetween. The first handle includes a switch activation element extending therefrom. A second handle depends from the housing and is aligned in registration with the first handle.

A switch is disposed in the second handle and is disposed in the actuation path of the switch activation element of the first handle, the switch configured for activation by the switch activation element of the first handle when the first handle is fully actuated relative to the second handle. A slider is disposed in the first handle and is movable between a first, in-line position configured to align the switch activation element with the switch to activate the jaw members upon full actuation of the first handle relative to the second handle and a second position wherein the switch activation element is misaligned with the switch such that full actuation of the first handle does not activate the jaw members.

In aspects according to the present disclosure, the switch is operably coupled to a depressible button extending from the housing and is configured to be selectively engaged by the switch activation element of the first handle when the slider is disposed in the first, in-line position.

In aspects according to the present disclosure, the slider is slidable within a channel defined within the first handle.

In aspects according to the present disclosure, the second handle includes an aperture defined therein configured to receive the switch activation element of the first handle upon full actuation of the first handle when the slider is disposed in the second position.

In aspects according to the present disclosure, the housing includes a latch assembly disposed therein configured to releasably lock the first handle relative to the second handle when the slider is disposed in the second position. In other aspects according to the present disclosure, the latch assembly is moveable between a first position wherein the latch assembly is aligned with a railway defined within the housing for selectively locking the first handle relative to the second handle upon actuation thereof and a second position wherein the latch assembly is misaligned with the railway and the first handle does not releasably lock relative to the second handle. In yet other aspects according to the present disclosure, the slider includes a rod operably coupled to the latch assembly, the rod configured to move the latch assembly between the first position and the second position upon movement of the slider. In still other aspects according to the present disclosure, the latch assembly is moveable about a pivot between the first and second positions,

In aspects according to the present disclosure, the latch assembly includes T-shape distal end configured to ride within the railway.

In aspects according to the present disclosure, the latch assembly is configured to lock the first handle relative to the second handle upon full actuation of the first handle relative to the second handle and initial release thereof and is configured to release the first handle from the railway upon and re-actuation of the first handle relative to the second handle and release thereof.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is a perspective view of an in-line, electrosurgical forceps according to an embodiment of the present disclosure including a housing, an elongated shaft, an end effector and an actuation handle;

FIG. 2A is an enlarged, perspective view of the end effector of FIG. 1 depicted with a pair of jaw members in an open configuration;

FIG. 2B is an enlarged, perspective view of the end effector of FIG. 1 depicted with the pair of jaw members in a closed configuration;

FIG. 3A is a perspective view of the end effector and elongated shaft of FIG. 1 with parts separated;

FIG. 3B is cross-sectional view taken along line 3B-3B of FIG. 3A showing a distal portion of the electrosurgical forceps of FIG. 1 depicting a tube guide;

FIG. 4 is a perspective view of a proximal portion of the forceps of FIG. 1 with a portion of the housing removed revealing internal components;

FIG. 5 is a partial, side view of a proximal portion of the forceps of FIG. 1;

FIG. 6A is an enlarged, internal perspective view of a slider configured to switch between in-line activation and manual activation of the electrosurgical forceps according to the present disclosure;

FIG. 6B is an enlarged, perspective view of a handle of the forceps of FIG. 6A exposing the internal features of the slider;

FIG. 6C is an enlarged, internal perspective view of a latch assembly of the forceps of FIG. 6A disposed in an activated position;

FIG. 6D is an enlarged view of the slider of FIG. 6B;

FIG. 7A is a perspective view of the forceps of FIG. 6A showing the slider de-activated or misaligned with an activation switch of the forceps;

FIG. 7B is a perspective view of the forceps of FIG. 6A showing the slider activated or aligned with an activation switch of the forceps allowing in-line activation;

FIG. 7C is an enlarged, internal view of the latch assembly de-activated when the slider is activated;

FIG. 8A is an enlarged, internal view of the latch assembly activated when the slider is de-activated in registration with a railway defined in the forceps for releasably locking the forceps when the handle is fully actuated;

FIG. 8B is an enlarged, internal view of the latch assembly de-activated when the slider is activated similar to FIG. 7C;

FIG. 8C is an enlarged, internal view of showing the range of motion of the latch assembly;

FIG. 9A is an enlarged, perspective view of the actuation handle of the electrosurgical forceps with the slider activated and showing a connection rod to the latch assembly;

FIG. 9B is a greatly-enlarged, front perspective view of the latching assembly and the components thereof;

FIG. 9C is a greatly-enlarged, rear perspective view of the latching assembly and the components thereof;

FIG. 10A is perspective view of the forceps with the actuation handle spaced relative to the activation switch with the slider positioned for in-line activation;

FIG. 10B is perspective view of the forceps with the actuation handle fully actuated with the slider activating the activation switch; and

FIG. 11 is perspective view of the forceps with the actuation handle slightly spaced relative to the activation switch with the slider de-activated allowing manual activation of the forceps;

### DETAILED DESCRIPTION

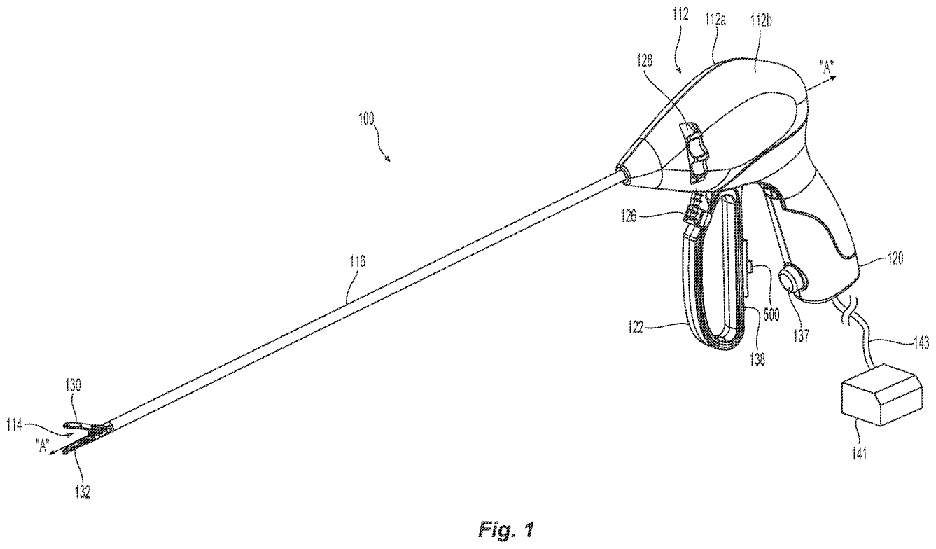

Referring initially to FIG. 1, an electrosurgical forceps 100 generally includes a housing 112 that supports various actuators thereon for remotely controlling an end effector 114 through an elongated shaft 116. Although this configuration is typically associated with instruments for use in laparoscopic or endoscopic surgical procedures, various aspects of the present disclosure may be practiced with traditional open instruments and in connection with endoluminal procedures as well. The housing 112 is constructed of a left housing half 112a and a right housing half 112b. The left and right designation of the housing halves 112a, 112b refer to the respective directions as perceived by an operator using the forceps 100. The housing halves 112a, 112b may be constructed of sturdy plastic, and may be joined to one another by adhesives, ultrasonic welding or other suitable assembly methods.

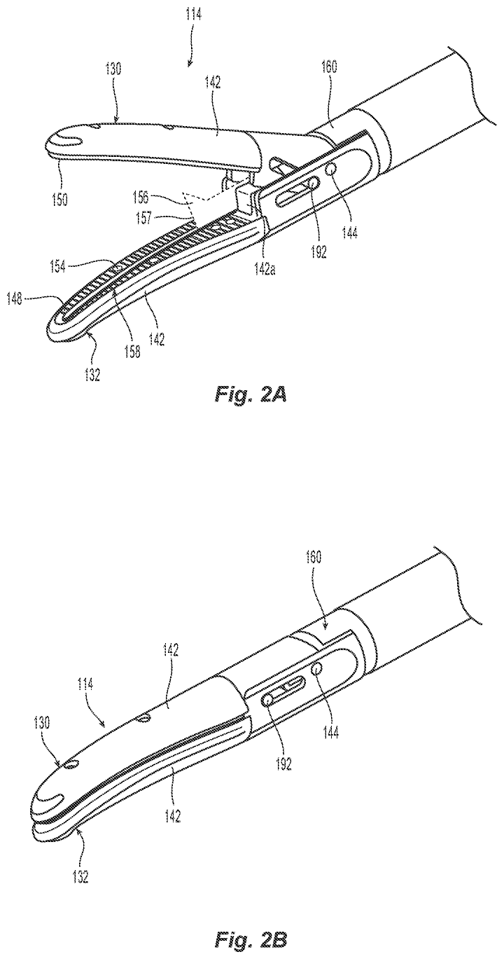

To mechanically control the end effector 114, the housing 112 supports a stationary handle 120, a movable handle 122, a trigger 126 and a rotation knob 128. The movable handle 122 is operable to move the end effector 114 between an open configuration (FIG. 2A) wherein a pair of opposed jaw members 130, 132 are disposed in spaced relation relative to one another, and a closed or clamping configuration (FIG. 2B) wherein the jaw members 130, 132 are closer together. Approximation of the movable handle 122 with the stationary handle 120 serves to move the end effector 114 to the closed configuration and separation of the movable handle 122 from the stationary handle 120 serves to move the end effector 114 to the open configuration. The trigger 126 is operable to extend and retract a knife blade 156 (see FIGS. 2A and 2B) through the end effector 114 when the end effector 114 is in the closed configuration. The rotation knob 128 serves to rotate the elongated shaft 116 and the end effector 114 about a longitudinal axis A-A extending through the forceps 114.

To electrically control the end effector 114, the stationary handle 120 supports a depressible button 137 thereon, which is operable by the user to initiate and terminate the delivery of electrosurgical energy to the end effector 114. The depressible button 137 is mechanically coupled to a switch (not shown) disposed within the stationary handle 120 and is engageable by a button activation post 138 extending from a proximal side of the moveable handle 122 upon proximal movement of the moveable handle 122 to an actuated or proximal position. The switch is in electrical communication with an electrosurgical generator 141 via suitable electrical wiring (not explicitly referenced) extending from the housing 112 through a cable 143 extending between the housing 112 and the electrosurgical generator 141. The generator 141 may include devices such as the LigaSure® Vessel Sealing Generator and the ForceTriad® Generator sold by Covidien. The cable 143 may include a connector (not shown) thereon such that the forceps 100 may be selectively coupled electrically to the generator 141.

Referring now to FIGS. 2A-2B, the end effector 114 may be moved from the open configuration (FIG. 2A) wherein tissue (not shown) is received between the jaw members 130, 132, and the closed configuration (FIG. 2B), wherein the tissue is clamped and treated. The jaw members 130, 132 pivot about a pivot pin 144 to move the end effector 114 to the closed configuration of FIG. 2B wherein the sealing plates 148, 150 provide a pressure to tissue grasped therebetween. In some embodiments, to provide an effective tissue seal, a pressure within a range between about 3 kg/cm² to about 16 kg/cm² and, desirably, within a working range of about 7 kg/cm² to about 13 kg/cm², may be applied to the tissue. Also, in the closed configuration, a separation or gap distance is maintained between the sealing plates 148, 150 by an array of stop members 154 (FIG. 2A) disposed on or adjacent the sealing plates 148, 150. The stop members 154 contact opposing surfaces on the opposing jaw member 130, 132 and prohibit further approximation of the sealing plates 148, 150. In some embodiments, to provide an effective tissue seal, an appropriate gap distance of about 0.001 inches to about 0.010 inches and, desirably, between about 0.002 inches to about 0.006 inches, may be provided. In some embodiments, the stop members 154 are constructed of a heat-resistant ceramic deposited onto the jaw members 130, 132. In other embodiments, the stop members 154 are constructed of an electrically non-conductive plastic molded onto the jaw members 130, 132, e.g., by a process such as overmolding or injection molding. The stop members 154 may define any suitable number, arrangement, and/or configuration, depending on a particular purpose.

The upper and lower jaw members 130, 132 are electrically coupled to cable 143, and thus to the generator 141 (e.g., via respective suitable electrical wiring extending through the elongated shaft 116) to provide an electrical pathway to a pair of electrically conductive, tissue-engaging sealing plates 148, 150 disposed on the lower and upper jaw members 132, 130, respectively. The sealing plate 148 of the lower jaw member 132 opposes the sealing plate 150 of the upper jaw member 130. In some embodiments, the sealing plates 148 and 150 are electrically coupled to opposite terminals, e.g., positive or active (+) and negative or return (−) terminals associated with the generator 141. Thus, bipolar energy may be provided through the sealing plates 148 and 150 to tissue. Alternatively, the sealing plates 148 and 150 may be configured to deliver monopolar energy to tissue. In a monopolar configuration, one or both sealing plates 148 and 150 deliver electrosurgical energy from an active terminal, e.g., (+), while a return pad (not shown) is placed generally on a patient and provides a return path to the opposite terminal, e.g., (−), of the generator 141. Each jaw member 130, 132 includes a jaw insert 140 and an insulator 142 that serves to electrically insulate the sealing plates 150, 148 from the jaw insert 140 of the jaw members 130, 132, respectively.

Electrosurgical energy may be delivered to the tissue through the electrically conductive seal plates 148, 150 to effect a tissue seal. Once a tissue seal is established, a knife blade 156 having a sharpened distal edge 157 may be advanced through a knife channel 158 defined in one or both jaw members 130, 132 to transect the sealed tissue. Although the knife blade 156 is depicted in FIG. 2A as extending from the elongated shaft 116 when the end effector 114 is in an open configuration, in some embodiments, extension of the knife blade 156 into the knife channel 158 when the end effector 114 is in the open configuration is typically prevented.

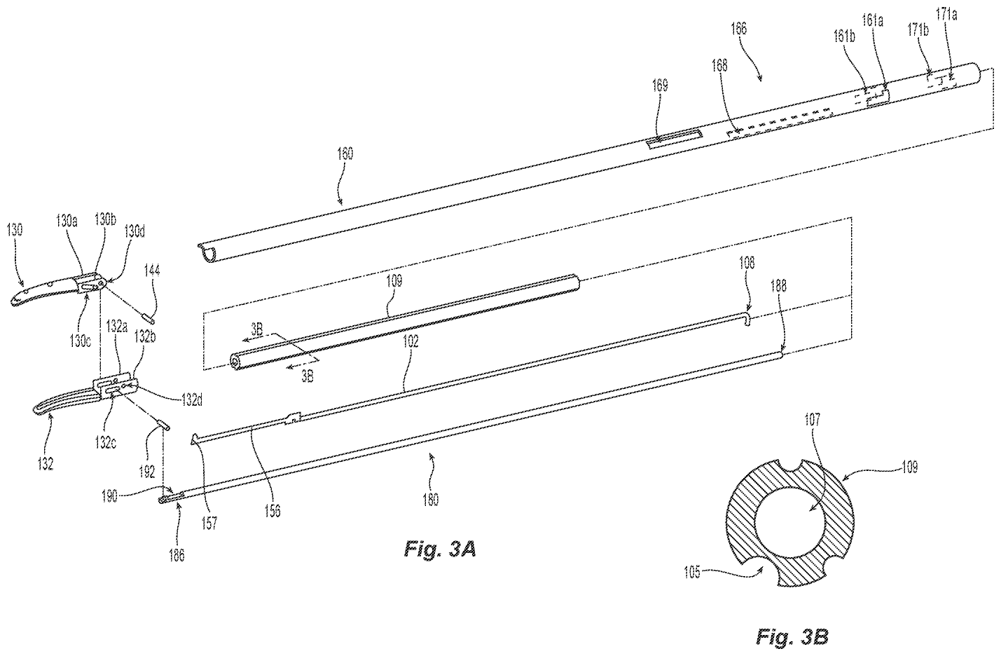

Referring to FIG. 3A, the elongated shaft 116 includes various longitudinal components that operatively couple the end effector 114 to the various actuators supported by the housing 112 (FIG. 1). An outer shaft member 160 defines an exterior surface of the elongated shaft 116 and houses other components therein as described below. The outer shaft member 160 is configured for longitudinal motion with respect to an inner actuation member 180 axially received within the outer shaft member 160. The inner actuation member 180 may be a rod, a shaft, a tube, folded metal, stamped metal, or other suitable structure. A proximal portion 166 of the outer shaft member 160 is configured for receipt within the housing 112 (FIG. 1), and includes features for operatively coupling the outer shaft member 160 to various elements of the housing 112. More specifically, the proximal portion 166 of the outer shaft member 160 includes, in order from distal to proximal, a longitudinal slot 169 to couple the outer shaft member 160 to the rotation knob 128, a longitudinal knife slot 168 defined therethrough, a pair of opposing distal locking slots 161a, 161b, and a pair of opposing proximal locking slots 171a, 171b.

A distal portion 186 of the inner actuation member 180 includes a longitudinal recess 190 defined therein that provides clearance for the pivot pin 144 and thus, permits longitudinal reciprocation of the pivot pin 144 (via longitudinal reciprocation of the outer shaft member 160) independent of the inner actuation member 180. Distally of the longitudinal recess 190, a cam pin 192 is mechanically coupled (e.g., via welding, friction-fit, laser welding, etc) to the distal portion 186 of the inner actuation member 180.

The pivot pin 144 extends through a proximal portion of each of the jaw members 130, 132 to pivotally support the jaw members 130, 132 at the distal end of the inner actuation member 180. A proximal portion of each of the jaw members 130, 132 includes two laterally spaced parallel flanges or “flags” 130a, 130b and 132a, 132b respectively, extending proximally from a distal portion of the jaw members 130 and 132 (FIG. 3A). A lateral cam slot 130c and a lateral pivot bore 130d extend through each of the flags 130a, 130b of the upper jaw member 130 (FIG. 3A). Similarly, a lateral cam slot 132c and a lateral pivot bore 132d extend through each of the flags 132a, 132b of the lower jaw member 132. The pivot bores 130d, 132d receive the pivot pin 144 in a slip-fit relation that permits the jaw members 130, 132 to pivot about the pivot pin 144 to move the end effector 114 between the open and closed configurations (FIGS. 2A and 2B, respectively).

A tube guide 109 is disposed within the outer shaft member 160 and includes a lumen 107 axially disposed therethrough. The inner actuation member 180 is received within the guide lumen 107, which serves to orient and align the inner actuation member 180 within the outer shaft member 160. The knife rod 102 is received within a longitudinal guide recess 105 formed in the outer surface of the guide tube 109. The guide recess 105 serves to guide longitudinal motion of the knife rod 102 within the outer shaft member 160 and to radially space the knife rod 102 from the inner actuation member 180 to prevent the inner actuation member 180 from interfering with reciprocal motion of the knife rod 102.

Rotation knob 128 imparts rotational motion to each of the components of the elongated shaft 116, and to the end effector 114, which is coupled thereto. The rotation knob 128 is supported in the housing 112 and, as shown in FIG. 1, extends radially outward from opposing sides of the housing 112 (only shown extending radially outward from housing half 112b).

End effector 114 is coupled to the distal end of the inner actuation member 180 by the cam pin 192. The cam pin 192 represents a longitudinally stationary reference for longitudinal movement of the outer shaft member 160 and the knife rod 102. The cam pin 192 extends through the flags 132a, 132b of the lower jaw member 132 and the flags 130a and 130b of the upper jaw member 130.

The outer shaft member 160 may be drawn proximally relative to the inner actuation member 180 and the cam pin 192 to move the end effector 114 to the closed configuration (see FIG. 2B). Since the longitudinal position of the cam pin 192 is fixed, and since the cam slot 130c is obliquely arranged with respect to the longitudinal axis A-A, proximal retraction of the outer shaft member 160 induces distal translation of the cam pin 192 through the cam slots 130c, 132c such that the jaw member 130 pivots toward jaw member 132 about the pivot pin 144. Conversely, when the end effector 114 is in the closed configuration, longitudinal translation of the outer shaft member 160 in a distal direction induces proximal translation of the cam pin 192 through the cam slots 130c, 132c such that jaw member 130 pivots away from jaw member 132 toward the open configuration.

The lower jaw member 132 is constructed of three major components: the jaw insert (not shown), the insulator 142, and the sealing plate 148. The flags 132a, 132b of the jaw member 132 define a proximal portion of the jaw insert and a generally u-shaped profile of the jaw insert extends distally to support the tissue engaging portion of the jaw member 132. Upper jaw member 130 includes the same three major components as lower jaw member 132, including sealing plate 150, jaw insert (not shown), and insulator 142, and is constructed in the same manner as lower jaw member 132. However, lower jaw member 132 is fixedly engaged, e.g., welded, to outer shaft member 160, while upper jaw member 130 is pivotable relative to lower jaw member 132 and outer shaft member 160 between the open and closed configurations.

In order to facilitate alignment of lower jaw member 132 with outer shaft member 160 during welding (or other suitable fixed engagement), the jaw insert and outer shaft member 160 may include complementary alignment features, e.g., a complementary recess (not explicitly shown) defined within jaw insert and a complementary protrusion (not explicitly shown) extending from outer shaft member 160. As an alternative to the unilateral configuration detailed above, both of the upper and lower jaw members 130, 132, respectively, may be pivotable relative to one another and outer shaft member 160, thus defining a bilateral configuration.

The insulator 142 of jaw members 130, 132 may be constructed of an electrically insulative plastic such as a polyphthalamide (PPA) (e.g., Amodel®), polycarbonate (PC), acrylonitrile butadiene styrene (ABS), a blend of PC and ABS, nylon, ceramic, etc. The insulator 142 may be overmolded onto the jaw insert in either a single-shot or a two-shot injection molding process such that each of the sealing plates 148, 150 are coupled to and in spaced relation with their respective jaw inserts 140.

In some embodiments, the insulator 142 on the lower jaw member 132 forms a tissue stop 142a extending therefrom adjacent to the knife channel 158 and proximal to the sealing plate 148. The tissue stop 142a serves to prevent tissue from entering the distal end of the outer shaft member 160 and to prevent splay of the flags 130a, 130b of the upper jaw member 130. In some embodiments, the tissue stop 142a may be formed by the insulator 142 on the upper jaw member 130 or on both the upper jaw member 130 and the lower jaw member 132. The tissue stop 142a may also serve to align the knife blade 156 as the knife blade 156 enters the knife channel 158 defined in the jaw members 130, 132. To this end, the surface of the tissue stop 142a extending along the path of the knife blade 156 may define a chamfered configuration to further facilitate alignment of the knife blade 156 as the knife blade 156 enters the knife channel 158.

The movable handle 122 may be manipulated to impart longitudinal motion to the outer shaft member 160, and the knife trigger 126 may be manipulated to impart longitudinal motion to the knife rod 102. As discussed above, longitudinal motion of the outer shaft member 160 serves to move the end effector 114 between the open configuration of FIG. 2A and the closed configuration of FIG. 2B, and longitudinal motion of the knife rod 102 serves to move knife blade 156 through knife channel 158 (FIG. 2A).

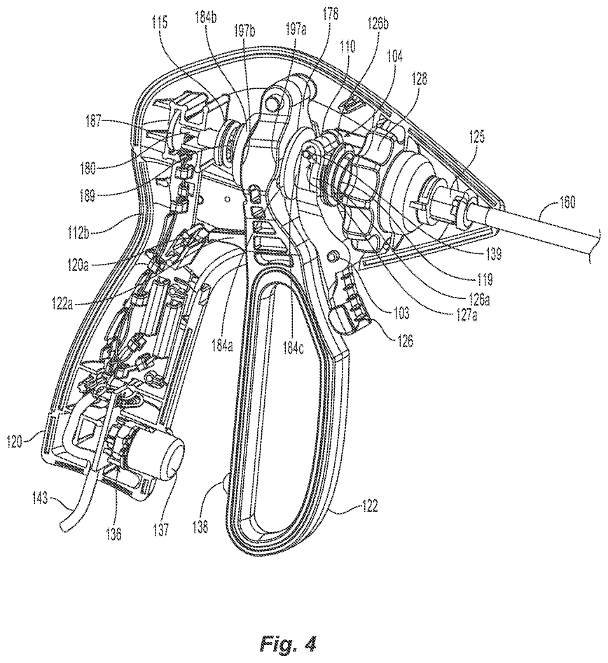

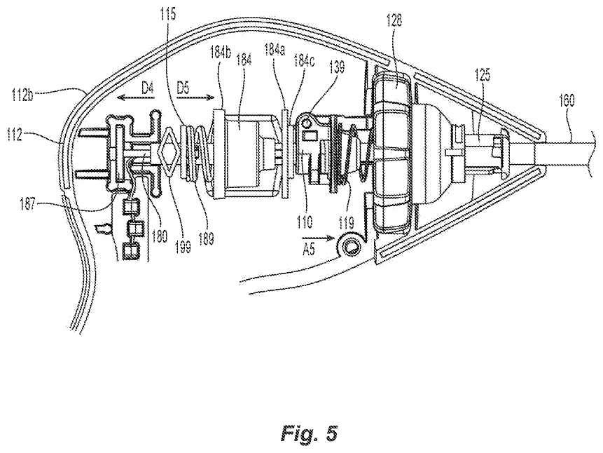

The movable handle 122 is operatively coupled to the outer shaft member 160 by a clevis 178 defined at an upper end of the movable handle 122 (FIG. 4). The clevis 178 is pivotally supported on the housing 112. The clevis 178 extends upwardly about opposing sides of a drive collar 184 (FIG. 5) supported on the outer shaft member 160 and includes rounded drive surfaces 197a and 197b thereon. Drive surface 197a engages a proximal-facing surface of a distal spring washer 184a and drive surface 197b engages a distal facing surface of a proximal rim 184b of the drive collar 184 (FIG. 5). The distal spring washer 184a engages a proximal facing surface of a distal spring stop 184c that, in turn, engages the opposing distal locking slots 161a, 161b (FIG. 3A) extending through the proximal portion 166 (FIG. 3A) of the outer shaft member 160 to couple the distal spring stop 184c to the outer shaft member 160. The drive surfaces 197a, 197b are arranged along the longitudinal axis A-A such that pivotal motion of the movable handle 122 induces corresponding longitudinal motion of the drive collar 184 (FIG. 5) along the longitudinal axis A-A.

Proximal longitudinal motion may be imparted to the outer shaft member 160 by pushing the proximal rim 184b of the drive collar 184 proximally with the movable handle 122 (FIG. 4) as indicated by arrow D4 (FIG. 5). A spring 189 is constrained between a proximal facing surface of the drive collar 184 and a proximal spring stop 115. The proximal spring stop 115 engages the opposing proximal locking slots 171a, 171b (FIG. 3A) extending through the proximal portion 166 (FIG. 3A) of the outer shaft member 160 to couple the proximal spring stop 115 to the outer shaft member 160. Thus, the proximal spring stop 115 serves as a proximal stop against which spring 189 compresses.

Distal longitudinal motion is imparted to the outer shaft member 160 by driving the drive collar 184 distally with the movable handle 122 (FIG. 4). Distal longitudinal motion of the drive collar 184 induces a corresponding distal motion of the outer shaft member 160 by virtue of the coupling of the drive collar 184 to opposing distal locking slots 181a, 181b extending through the proximal portion 166 of the outer shaft member 160 (FIG. 3A).

Proximal longitudinal motion of the outer shaft member 160 draws jaw member 132 proximally such that the cam pin 192 advances distally to pivot jaw member 130 toward jaw member 132 to move the end effector 114 to the closed configuration. Once the jaw members 130 and 132 are closed, the outer shaft member 160 essentially bottoms out (i.e., further proximal movement of the outer shaft member 160 is prohibited since the jaw members 130, 132 contact one another). Further proximal movement of the movable handle 122 (FIG. 4), however, will continue to move the drive collar 184 proximally. This continued proximal movement of the drive collar 184 further compresses the spring 189 to impart additional force to the outer shaft member 160, which results in additional closure force applied to tissue grasped between the jaw members 130, 132 (see FIG. 2B).

Referring again to FIG. 4, the trigger 126 is pivotally supported in the housing 112 about a pivot boss 103 protruding from the trigger 126. The trigger 126 is operatively coupled to the knife rod 102 by a knife connection mechanism 104 such that pivotal motion of the trigger 126 induces longitudinal motion of the knife rod 102. The knife connection mechanism 104 includes upper flanges 126a, 126b of the trigger 126 and a knife collar 110.

As the moveable handle 122 is moved to a fully actuated or proximal position of, the button activation post 138 depresses the depressible button 137, thereby activating the switch disposed within the stationary handle 120 to initiate the delivery of electrosurgical energy to the end effector 114 to generate a tissue seal.

As the movable handle 122 is moved from an intermediate position to a fully actuated or proximal position of, the pressure applied by the jaw members 130, 132 is increased. As the movable handle 122 pivots further the spring 189 is compressed against the proximal spring stop 115, and a tensile force is transmitted through the outer shaft member 160 to the jaw members 130, 132. The tensile force supplied by the spring 189 ensures that the jaw members 130, 132 apply an appropriate pressure to effect a tissue seal.

When the movable handle 122 is in the fully actuated or proximal position, the knife trigger 126 may be selectively moved to a proximal position to advance the knife blade 156 distally through knife channel 158.

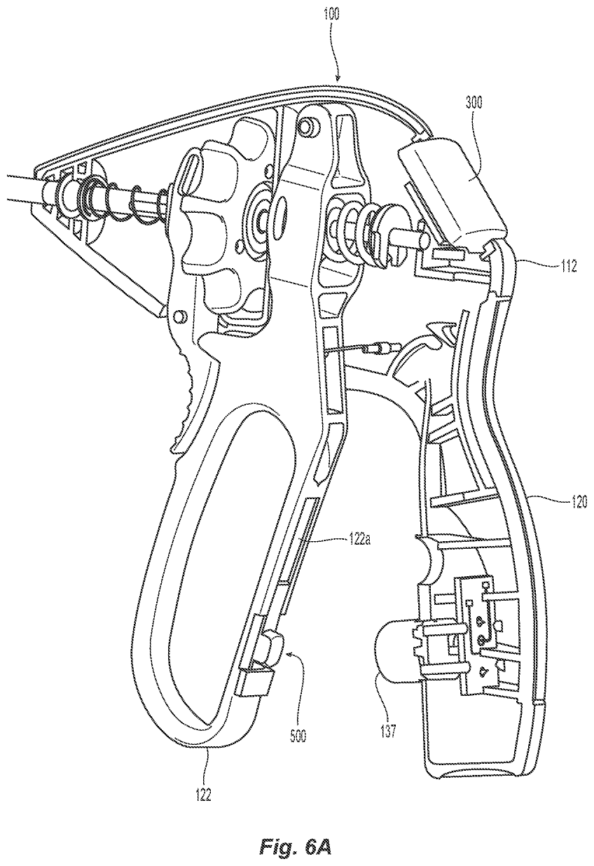

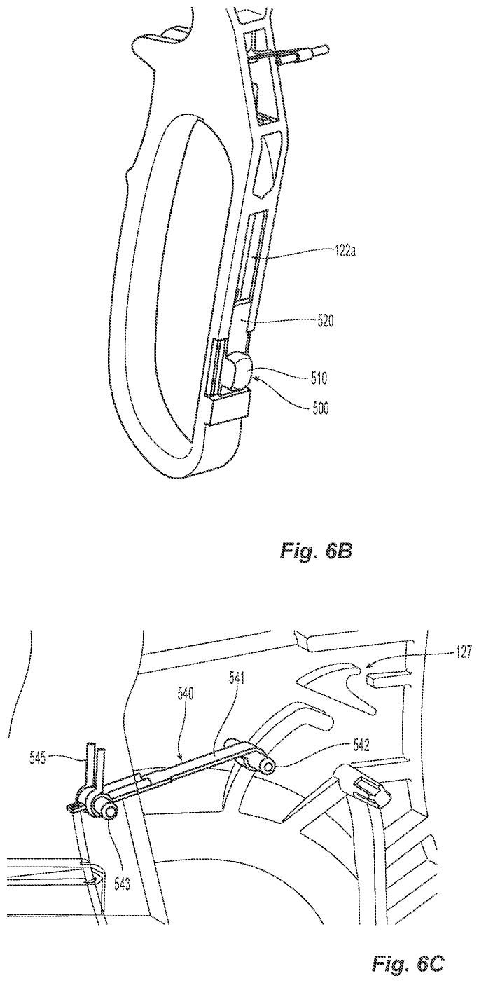

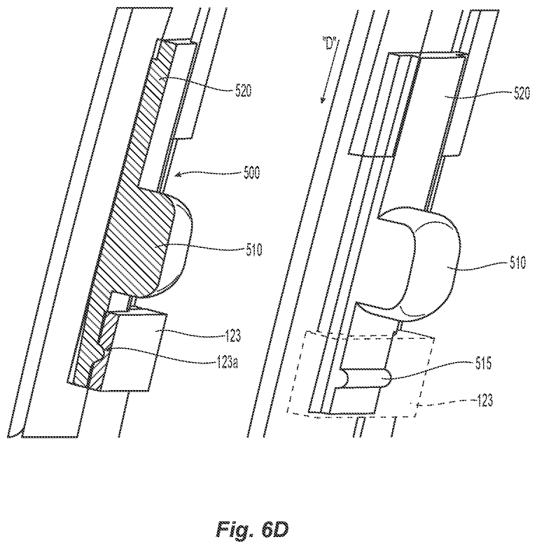

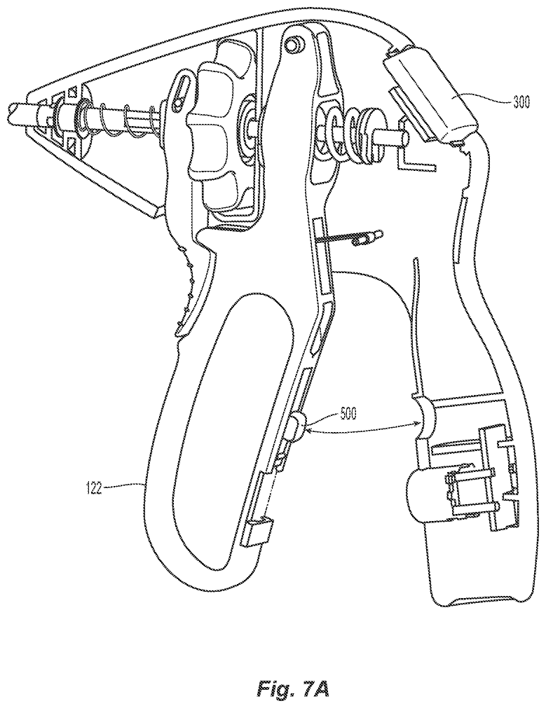

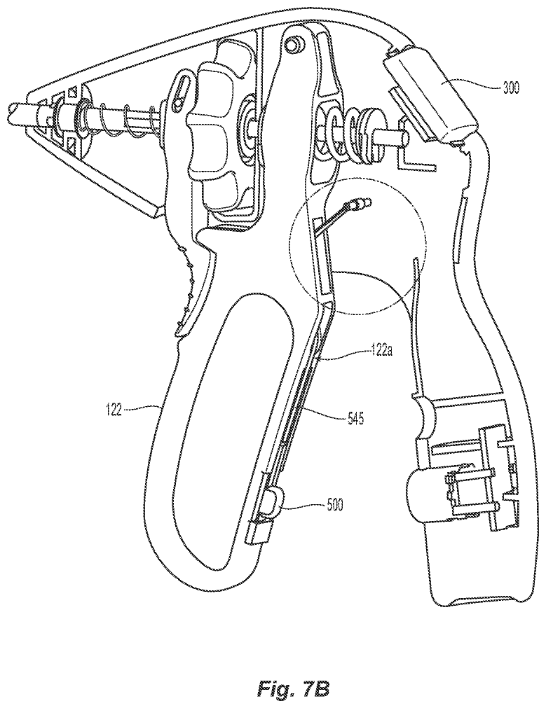

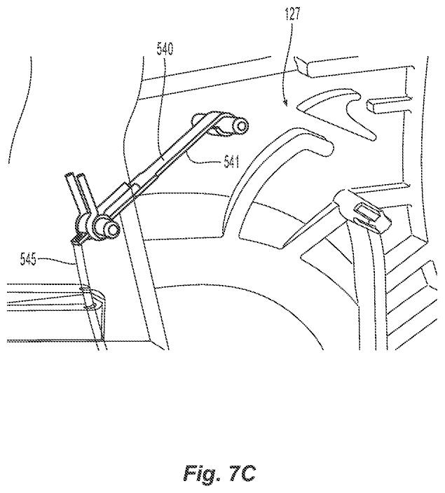

FIGS. 6A-7D show one embodiment of an in-line activation slider 500 for the electrosurgical forceps 100 according to the present disclosure enabling a surgeon to selectively choose between an in-line activation mode wherein the handle 122 activates the switch 137 through the range of motion and a more traditional mode which allows a surgeon to grasp and then activate the jaw members 130, 132. More particularly, in-line activation slider (hereinafter “slider 500”) is disposed on handle 122 of the forceps 100 in a convenient location for easy access thereto with a surgeon's thumb or finger. As shown, slider 500 is disposed on the proximal-facing side of handle 122 facilitating access for either right or left-handed surgeons (FIGS. 6A and 6B).

Slider 500 includes a slide plate 520 having a knob 510 extending therefrom to facilitate engagement with a surgeon's finger. As explained in more detail below, slide plate 520 is slidingly disposed in an elongated channel 122a defined in the proximal face of handle 122 (FIG. 6B) and is moveable therealong upon actuation of knob 510 between an in-line position enabling in-line activation of the forceps 100 upon actuation of handle 122 and a traditional position enabling grasping of tissue between jaw members 130, 132 and subsequent activation of the forceps 100.

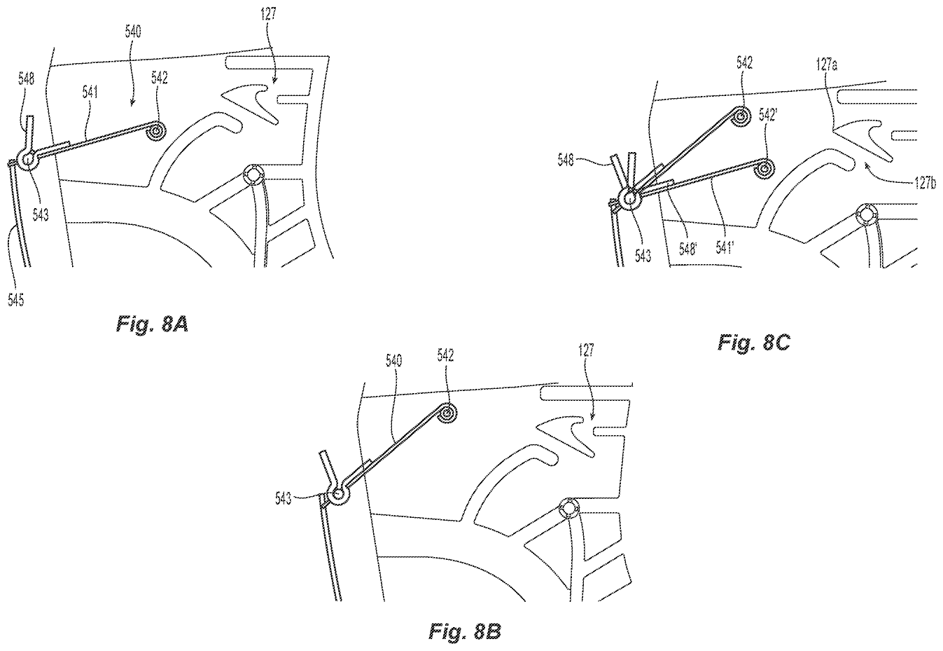

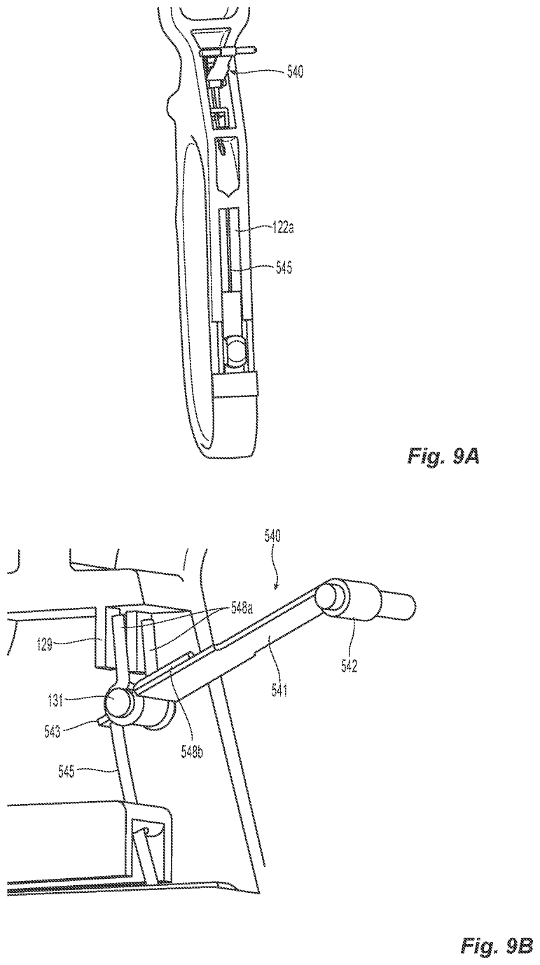

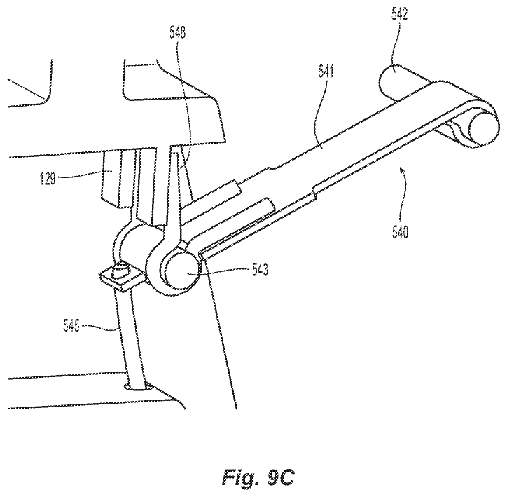

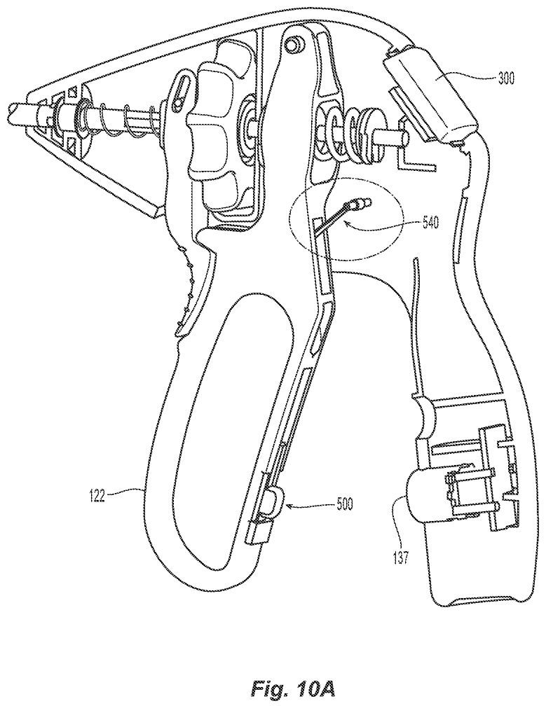

In order to facilitate in-line electrosurgical activation, the surgeon moves knob 510 within channel 122a to the in-line position (shown as down arrow “D” in the various figures, e.g., FIG. 6D) which orients the knob 510 in angular relation with switch 137. The slider plate 520 includes a detent 515 thereon that is configured to operably engage or snap-fit the slider plate 520 within a pocket 123 defined in handle 122. This ensures that the slider plate 520 is temporality locked in place in snap-fit engagement during use. Other mechanical interfaces are also contemplated. Once locked in place, the user simply actuates handle 122 to grasp tissue and during the range of motion activates the jaw members 130, 132 as described above. Various haptic or audible devices (not shown) may be employed to alert the surgeon prior to electrical activation. As described in more detail below with respect to FIGS. 8A-9C, moving the slider plate 520 to the in-line position deactivates a latch assembly 540.

In order to facilitate a more traditional approach to electrosurgical activation, the surgeon moves knob 510 within channel 122a to the second or more traditional, manual activation position (shown as up arrow “U” in the FIG. 11) which orients the knob 510 out of angular relation with switch 137. One or more mechanical features may be employed to lock the knob in the second position. As best shown in FIG. 11, knob 510 aligns with an aperture 120a defined in handle 120 such that, upon manual actuation of handle 122 relative to handle 120, the knob 510 is received unencumbered therein. Likewise, channel 122a defined in handle 122 is dimensioned to receive switch 137 unencumbered therein, i.e., switch 137 does not activate upon full compression of handle 122 against handle 120.

Once the knob 510 locked in place, the user simply actuates handle 122 to grasp tissue and, once grasped, subsequently activates the jaw members 130, 132 via switch 300 (FIG. 6A) as described above. Various haptic or audible devices (not shown) may be employed to alert the surgeon that the tissue is fully grasped under the correct sealing pressure prior to electrical activation. Alternatively and as shown in FIGS. 8A-9C described in more detail below, moving the slider plate 520 up “U” to the second position activates the latch assembly 540 so that the handle 122 may be locked in a grasped orientation for subsequent electrical activation of forceps 100.

Turning now to FIGS. 8A-9C, latch assembly 540 is disposed within the housing 112 of the forceps 100 generally between handles 122 and 120. Latch assembly 540 includes an elongated arm 541 having proximal and distal ends 543, 542, respectively. Proximal end 543 operably couples to a torsion spring 548 mounted within housing 112. Torsion spring 548 includes opposing arms 548a, 548b that bias one another upon movement thereof (arms 548a and 548b may each be a pair of arms as shown for stability purposes during the range of movement thereof). A flange 129 disposed in housing 112 (FIG. 9B) provides the bias between the arms 548a, 548b.

The distal end 542 of the latch assembly 540 is generally T-shaped and is configured to ride within a railway 127 defined in handle 120. Railway 127 includes a track for the T-shaped distal end 542 to lock and unlock the handle 122 upon repeated use when grasping ands sealing tissue with a more traditional surgical approach. More particularly and as shown in FIGS. 8A-8C), when oriented for traditional use (e.g., the slider plate 520 is disposed in the second position) upon movement of the handle 122 relative to handle 120, the T-shaped distal end 542 is introduced into a distal end 127a of railway 127 and then directed to a rest 127b upon release of the handle 122 (FIG. 8C). The bias of the torsion spring 548 directs the T-shaped distal end around the railway 127 and into the rest 127b to lock the handle 122 relative to handle 120. Upon regrasp of the handle 122, the T-shaped distal end is redirected away from rest 127b and thereafter released to allow the handle 122 to open.

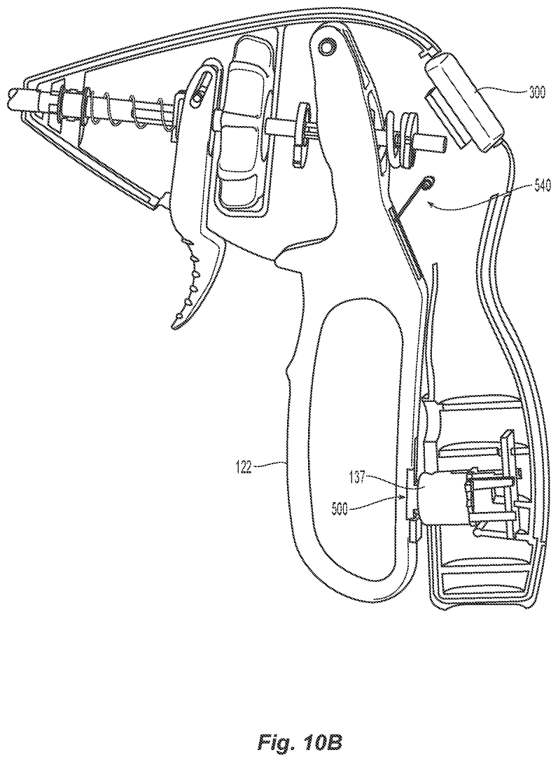

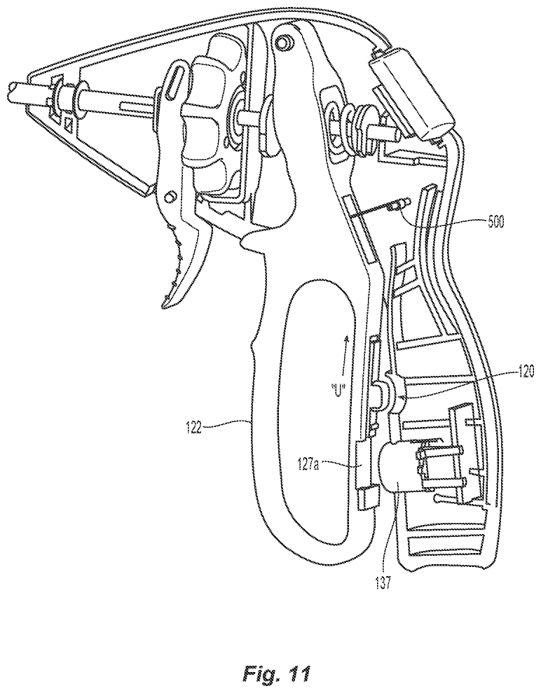

To reconfigure the forceps for in-line use and as mentioned above, the surgeon moves slider plate 520 via knob 510 to the in-line position down “D” which aligns the knob 510 with switch 137. At the same time, a rod 545 attached at one end to the proximal end 543 of arm 541 of latch assembly 540 and at the other end to the slide plate 520 pivots the latch assembly 540 about a pivot 131 to misalign the latch assembly 540 with the railway 127 such that the latch assembly 540 does not lock upon actuation of handle 122 relative to handle 120 (FIGS. 10A, 10B). In other words, moving the slider 500 to the in-line position, moves the rod 545 to pivot the latch assembly 520 away from the railway 127. Moving the slider 500 to the second position, pivots the latch assembly 520 to ride the railway 127 to lock the handles 122, 120 relative to one another for subsequent electrical activation.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

Although the foregoing disclosure has been described in some detail by way of illustration and example, for purposes of clarity or understanding, it will be obvious that certain changes and modifications may be practiced within the scope of the appended claims.

What is claimed is:

1. A surgical instrument, comprising:
a housing including an elongated shaft having an end effector assembly disposed at a distal end thereof, the end effector including first and second jaw members configured to treat tissue upon electrical activation thereof;
a first handle movable relative to the housing and configured to move at least one of the first or second jaw members relative to the other of the first or second jaw members to grasp tissue therebetween, the first handle including a switch activation element extending therefrom;
a switch disposed on the housing and in the actuation path of the first handle, the switch configured for activation by the first handle when the first handle is fully actuated relative to the housing; and
a slider disposed in the first handle and movable between a first, in-line position configured to align the switch activation element with the switch to activate the jaw members upon full actuation of the first handle relative to the housing and a second position wherein the switch activation element is misaligned with the switch such that full actuation of the first handle does not activate the jaw members.

2. The surgical instrument according to claim 1, wherein the switch is operably coupled to a depressible button extending from the housing and configured to be selectively engaged by the switch activation element of the first handle when the slider is disposed in the first, in-line position.

3. The surgical instrument according to claim 1, wherein the slider is slidable within a channel defined within the first handle.

4. The surgical instrument according to claim 1, wherein the housing includes a second handle depending therefrom configured to support the switch therein.

5. The surgical instrument according to claim 4, wherein the second handle includes an aperture defined therein configured to receive the switch activation element of the first handle upon full actuation of the first handle when the slider is disposed in the second position.

6. The surgical instrument according to claim 1, wherein the housing includes a latch assembly disposed therein configured to releasably lock the first handle relative to the housing when the slider is disposed in the second position.

7. The surgical instrument according to claim 6, wherein the latch assembly is moveable between a first position wherein the latch assembly is aligned with a railway defined within the housing for selectively locking the first handle relative to the housing upon actuation thereof and a second position wherein the latch assembly is misaligned with the railway and the first handle does not releasably lock relative to the housing.

8. The surgical instrument according to claim 7, wherein the slider includes a rod operably coupled to the latch assembly, the rod configured to move the latch assembly between the first position and the second position upon movement of the slider.

9. A surgical instrument, comprising:
a housing including an elongated shaft having an end effector assembly disposed at a distal end thereof, the end effector including first and second jaw members configured to treat tissue upon electrical activation thereof;
a first handle movable relative to the housing and configured to move at least one of the first or second jaw members relative to the other of the first or second jaw members to grasp tissue therebetween, the first handle including a switch activation element extending therefrom;
a second handle depending from the housing and aligned in registration with the first handle;
a switch disposed in the second handle and in the actuation path of the switch activation element of the first handle, the switch configured for activation by the switch activation element of the first handle when the first handle is fully actuated relative to the second handle; and
a slider disposed in the first handle and movable between a first, in-line position configured to align the switch activation element with the switch to activate the jaw members upon full actuation of the first handle relative to the second handle and a second position wherein the switch activation element is misaligned with the switch such that full actuation of the first handle does not activate the jaw members.

10. The surgical instrument according to claim 9, wherein the switch is operably coupled to a depressible button extending from the housing and configured to be selectively engaged by the switch activation element of the first handle when the slider is disposed in the first, in-line position.

11. The surgical instrument according to claim 9, wherein the slider is slidable within a channel defined within the first handle.

12. The surgical instrument according to claim 9, wherein the second handle includes an aperture defined therein configured to receive the switch activation element of the first handle upon full actuation of the first handle when the slider is disposed in the second position.

13. The surgical instrument according to claim 9, wherein the housing includes a latch assembly disposed therein configured to releasably lock the first handle relative to the second handle when the slider is disposed in the second position.

14. The surgical instrument according to claim 13, wherein the latch assembly is moveable between a first position wherein the latch assembly is aligned with a railway defined within the housing for selectively locking the first handle relative to the second handle upon actuation thereof and a second position wherein the latch assembly is misaligned with the railway and the first handle does not releasably lock relative to the second handle.

15. The surgical instrument according to claim 14, wherein the slider includes a rod operably coupled to the latch assembly, the rod configured to move the latch assembly between the first position and the second position upon movement of the slider.

16. The surgical instrument according to claim 15, wherein the latch assembly is moveable about a pivot between the first and second positions.

17. The surgical instrument according to claim 14, wherein the latch assembly includes T-shape distal end configured to ride within the railway.

18. The surgical instrument according to claim 14, wherein the latch assembly is configured to lock the first handle relative to the second handle upon full actuation of the first handle relative to the second handle and initial release thereof and configured to release the first handle from the railway upon and re-actuation of the first handle relative to the second handle and release thereof.
